(19) United States
(12) Patent Application Publication
(10) Pub. No.: US 2025/0099813 A1
(43) Pub. Date: Mar. 27, 2025

(54) PHYSICAL FITNESS MEASUREMENT METHOD USING AUGMENTED REALITY INTERACTIVE SPORTS DEVICE

(71) Applicant: Twohands Interactive Inc., Busan (KR)

(72) Inventor: Wangyun YOO, Busan (KR)

(21) Appl. No.: 18/889,478

(22) Filed: Sep. 19, 2024

(30) Foreign Application Priority Data
Sep. 24, 2023 (KR) 10-2023-0127582

(51) Int. Cl. A63B 24/00; A61B 5/00; A63B 71/06

(52) U.S. Cl. CPC A63B 24/0062; A61B 5/4884; A63B 71/0622; A63B 2024/0065; A63B 2071/0638; A63B 2220/805

(57) ABSTRACT

A physical fitness measurement method using an augmented reality interactive sports device is proposed. The physical fitness measurement method is realized by the augmented reality interactive sports device that projects an image on a floor surface, tracks a user's motion by using LiDAR sensor units, and controls interaction between content and the user, and includes displaying distance measurement-type, count measurement-type, or time measurement-type physical fitness measurement content on the floor surface through the augmented reality interactive sports device, using the LiDAR sensor units that perform laser scanning of a plane at a predetermined height from the floor surface, so as to detect foot or hand positions of the user and determining event occurrences according to the physical fitness measurement content and the foot or hand positions, and measuring a distance, count, or time of each event occurrence determined according to the physical fitness measurement content.

### CROSS-REFERENCE TO RELATED APPLICATION

The present application claims priority to Korean Patent Application No. 10-2023-0127582, filed on Sep. 24, 2023, the entire contents of which are incorporated herein for all purposes by this reference.

### BACKGROUND OF THE INVENTION

### Field of the Invention

The present disclosure relates to a physical fitness measurement method and, more particularly, to a physical fitness measurement method using an augmented reality interactive sports device.

### Description of the Related Art

The contents described in this section simply provide background information on an exemplary embodiment of the present disclosure, and do not constitute the related art.

Physical fitness measurement is a quantitative measurement and determination of fitness from various perspectives and methods in order not only to increase efficiency without difficulty while engaging in physical exercise, labor, or the like but also to correct various defects in body posture and improve health effects. Specific physical fitness measurement items vary depending on the life cycle of adolescence, adulthood, and the elderly, but in general, cardiorespiratory endurance, muscular strength, muscular endurance, flexibility, agility, quickness, and coordination, etc. are assessed.

Such physical fitness measurement helps determine current physical conditions and establish future exercise directions, etc., and the health and physical fitness status may be monitored through regular physical fitness measurement. However, a typical adult does not have many opportunities for such physical fitness measurement, thereby making it difficult to objectively understand one's physical conditions.

Accordingly, National Physical Fitness 100 is being operated as a public sports welfare service that measures and assesses an individual's physical fitness and provides exercise consultation and prescriptions. However, this service has a limitation of low accessibility because of requiring a search for a place where the corresponding service is available and a visit to the place in person for measuring his or her physical fitness.

Meanwhile, as a related art to the present disclosure, Korean Patent No. 10-2346069 (invention title: METHOD AND SYSTEM FOR MEASURING PHYSICAL FITNESS THROUGH NON-FACE-TO-FACE PHYSICAL ACTIVITY, registration date: Dec. 28, 2021), etc. have been disclosed.

The above-described background art is technical information that the inventor possessed for derivation of the present disclosure or acquired in a derivation process of the present disclosure, and is not necessarily known technology disclosed to the general public prior to filing the embodiment of the present disclosure.

### SUMMARY OF THE INVENTION

The present disclosure is proposed to solve the above-described problems of previously proposed methods, and an objective of the present disclosure is to provide a physical fitness measurement method using an augmented reality interactive sports device capable of displaying distance measurement-type, count measurement-type, or time measurement-type physical fitness measurement content on a floor surface through the augmented reality interactive sports device and determining event occurrences according to foot or hand positions by using LiDAR sensors, so as to measure a distance, count, or time of each event occurrence, whereby a user's physical fitness may be conveniently measured by using digital technology, physical fitness may be repeatedly and autonomously measured, and changes in the physical fitness may be monitored.

In addition, another objective of the present disclosure is to provide a physical fitness measurement method using an augmented reality interactive sports device capable of performing laser scanning of heights of foot positions at a certain height from a floor surface, so as to determine event occurrences, thereby measuring lower body abilities such as a long jump, a round-trip run, etc. in an easy and fun way.

However, the technical problems to be solved by the present disclosure is not limited to the technical problems as described above, and other technical problems may exist. Even though not explicitly mentioned, the present disclosure naturally includes other objectives or effects, which may be identifiable from the problem solutions or embodiments.

According to the features of the present disclosure to achieve the above described objectives, there is provided a physical fitness measurement method using an augmented reality interactive sports device 100, the physical fitness measurement method being realized by the augmented reality interactive sports device 100 that projects an image on a floor surface, tracks a user's motion by using LiDAR sensor units 130a and 130b, and controls interaction between content and the user, and the physical fitness measurement method including: step (1) of displaying distance measurement-type, count measurement-type, or time measurement-type physical fitness measurement content on the floor surface through the augmented reality interactive sports device 100; step (2) of using the LiDAR sensor units 130a and 130b that perform laser scanning of a plane at a predetermined height from the floor surface, so as to detect foot or hand positions of the user and determining event occurrences according to the physical fitness measurement content and the foot or hand positions; and step (3) of measuring a distance, count, or time of each event occurrence determined according to the physical fitness measurement content.

Preferably, a reference position and a distance measurement direction may be displayed on the floor surface, so as to display the distance measurement-type physical fitness measurement content in step (1), each event occurrence may be determined when the user's feet are detected in the distance measurement direction in step (2), and a distance between a first position of the user's feet detected at the reference position and a second position of the user's feet detected at a time of each event occurrence may be measured, so that any one selected from a group including a standing long jump distance and a leg stretch distance may be measured in step (3).

More preferably, the physical fitness measurement method may further include: after step (3), step (4) of displaying an event occurrence image at the second position, displaying an arrow on the floor surface in a direction from the first position of the user's feet to the second position, and displaying the distance measured in step (3) around the arrow.

Preferably, at least two or more sprites having shapes different from each other may be displayed on the floor surface where the user's feet or hands will be positioned, so as to display the count measurement-type physical fitness measurement content in step (1), the event occurrences may be determined in the corresponding sprites when the user's detected foot or hand positions correspond to the sprites in step (2), and the number of event occurrences in the sprites may be measured in step (3).

More preferably, in step (2), at least one of the forms, shapes, and colors of the sprites determined as to have the event occurrences may be displayed to change, so as to indicate that the user's feet or hands correspond to the corresponding sprites.

More preferably, in step (3), the number of event occurrences during a predetermined time period may be measured, and any one selected from a group including the number of round-trip runs and the number of burpee tests may be measured.

Preferably, sprites may be displayed on the floor surface where the user's feet or hands will be positioned, so as to display the time measurement-type physical fitness measurement content in step (1), the event occurrences may be determined in the corresponding sprites when the user's detected foot or hand positions correspond to the sprites in step (2), and a time between a first time point when the user's detected foot or hand positions in the sprites begin to be undetected and a second time point when the user's feet or hands are not detected and then detected again may be measured, so as to measure any one selected from a group including a one-legged stand time in step (3).

More preferably, in step (2), at least one of sizes, shapes, and colors of the sprites determined as to have the event occurrences may be displayed to change, so as to indicate that the user's feet or hands correspond to the corresponding sprites.

According to the physical fitness measurement method using the augmented reality interactive sports device proposed in the present disclosure, there is an effect of displaying distance measurement-type, count measurement-type, or time measurement-type physical fitness measurement content on a floor surface through the augmented reality interactive sports device and determining event occurrences according to foot or hand positions by using LiDAR sensors, so as to measure a distance, count, or time of each event occurrence, whereby a user's physical fitness may be conveniently measured by using digital technology, physical fitness may be repeatedly and autonomously measured, and changes in the physical fitness may be monitored.

In addition, according to the physical fitness measurement method using the augmented reality interactive sports device proposed in the present disclosure, there is another effect of performing laser scanning of heights of foot positions at a certain height on a floor surface, so as to determine event occurrences, thereby measuring lower body abilities such as a long jump, a round-trip run, etc. in an easy and fun way.

In addition, the various and advantageous strong points and effects of the present disclosure are not limited to the above-described content, and will be more easily understood in the process of describing the specific embodiment of the present disclosure.

### BRIEF DESCRIPTION OF THE DRAWINGS

FIG. 1 is a view illustrating a configuration of a physical fitness measurement device using an augmented reality interactive sports device according to an exemplary embodiment of the present disclosure.

FIG. 2 is a view illustrating the appearance of an augmented reality interactive sports device used in a physical fitness measurement method using the augmented reality interactive sports device according to the exemplary embodiment of the present disclosure.

FIG. 3 is a view illustrating a flow of the physical fitness measurement method using the augmented reality interactive sports device according to the exemplary embodiment of the present disclosure.

FIG. 4 is a view illustrating types of physical fitness measurement content in the physical fitness measurement method using the augmented reality interactive sports device according to the exemplary embodiment of the present disclosure.

FIG. 5 is a view illustrating a flow of a physical fitness measurement method according to distance measurement-type physical content in the physical fitness measurement fitness measurement method using the augmented reality interactive sports device according to the exemplary embodiment of the present disclosure.

FIG. 6 is a view illustrating an example of an appearance of standing long jump content displayed on a floor surface in the physical fitness measurement method using the augmented reality interactive sports device according to the exemplary embodiment of the present disclosure.

FIG. 7 is a view illustrating an example of an appearance of leg stretch content displayed on a floor surface in the physical fitness measurement method using the augmented reality interactive sports device according to the exemplary embodiment E the present disclosure.

FIG. 8 is a view illustrating a flow of a physical fitness measurement method according to count measurement-type physical fitness measurement content in the physical fitness measurement method using the augmented reality interactive sports device according to the exemplary embodiment of the present disclosure.

FIG. 9 is a view illustrating an example of an appearance of round-trip run content displayed on a floor surface in the physical fitness measurement method using the augmented reality interactive sports device according to the exemplary embodiment of the present disclosure.

FIG. 10 is a view illustrating an example of an appearance of burpee test content displayed on a floor surface in the physical fitness measurement method using the augmented reality interactive sports device according to the exemplary embodiment of the present disclosure.

FIG. 11 is a view illustrating an example of an appearance of square content displayed on a floor surface in the physical fitness measurement method using the augmented reality interactive sports device according to the exemplary embodiment of the present disclosure.

FIG. 12 is a view illustrating a flow of a physical fitness measurement method according to time measurement-type physical fitness measurement content in the physical fitness measurement method using the augmented reality interactive sports device according to the exemplary embodiment of the present disclosure.

FIG. 13 is a view illustrating an example of an appearance of one-legged stand content displayed on a floor surface in the physical fitness measurement method using the augmented reality interactive sports device according to the exemplary embodiment of the present disclosure.

### DETAILED DESCRIPTION OF THE INVENTION

Hereinafter, an exemplary embodiment of the present disclosure will be described in detail with reference to the accompanying drawings so that those skilled in the art may easily implement the present disclosure. However, the present disclosure is not limited to the exemplary embodiment described herein and may be embodied in many different forms. In addition, in order to clearly describe the present disclosure in the drawings, parts irrelevant to the description are omitted, and like reference numerals designate like elements throughout the present specification.

Throughout the specification, when a part is said to be “connected” to another part, an expression such as “connected” is intended to include not only “directly connected” but also “indirectly connected” having a different component in the middle thereof. In addition, it will be further understood that, when a part is said to “include” or “comprise” a certain component, it means that it may further include or comprise other components, but does not exclude other components unless the context clearly indicates otherwise, and does not preclude the presence or addition of one or more other features, integers, steps, operations, elements, components, and/or combinations thereof.

The following exemplary embodiment is detailed descriptions for better understanding of the present disclosure, and does not limit the scope of the present disclosure. Therefore, an embodiment of the same scope that performs the same functions as those of the present disclosure will also fall within the scope of the present disclosure.

In addition, each component, process, progress, method, or the like included in the exemplary embodiment of the present disclosure may be shared within a range that does not contradict each other technically.

In addition, in the present disclosure, some of the operations or functions described as being performed by a terminal, apparatus, or device may be performed instead by a server connected to the terminal, apparatus, or device. Likewise, some of the operations or functions described as being performed by the server may also be performed by the terminal, apparatus, or device connected to the corresponding server.

In particular, a means for executing a system according to the exemplary embodiment of the present disclosure may be an application or a web server, and a terminal, which is a means for reading a recording medium on which the application or web server is recorded, may include not only a general PC such as a general desktop or laptop computer, but also a mobile terminal such as a smartphone and a tablet PC.

Hereinafter, the exemplary embodiment of the present disclosure will be described in detail with reference to the accompanying drawings.

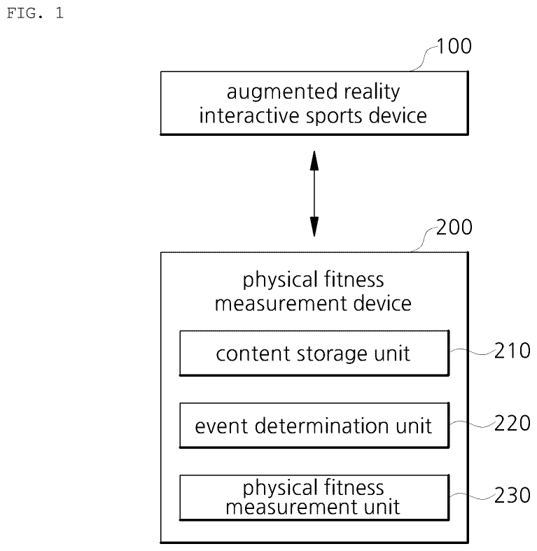

FIG. 1 is a view illustrating a configuration of a physical fitness measurement device 200 using an augmented reality interactive sports device 100 according embodiment of the present disclosure. As shown in FIG. 1, according to the exemplary embodiment of the present disclosure, the physical fitness measurement device 200 using the augmented reality interactive sports device 100 may be configured to include: a content storage unit 210 configured to transmit and receive data with the augmented reality interactive sports device 100 that projects an image on a floor surface, tracks a user's motion by using LiDAR sensor units 130a and 130b, and controls interaction between content and the user, and store distance measurement-type, count measurement-type, or time measurement-type physical fitness measurement content on the floor surface provided through the augmented reality interactive sports device 100; an event determination unit 220 configured to detect foot or hand positions of the user by using the LiDAR sensor units 130a and 130b performing laser scanning of a plane at a predetermined height from the floor surface, and determine event occurrences according to the physical fitness measurement content and the foot or hand positions; and a physical fitness measurement unit 230 configured to measure a distance, count, or time of each event occurrence determined according to the physical fitness measurement content. According to the exemplary embodiment of the present disclosure, the physical fitness measurement device 200 using the augmented reality interactive sports device 100 may be built into the augmented reality interactive sports device 100 or communicate with the augmented reality interactive sports device 100 by wire or wirelessly, so as to analyze the user's gait and provide analysis results.

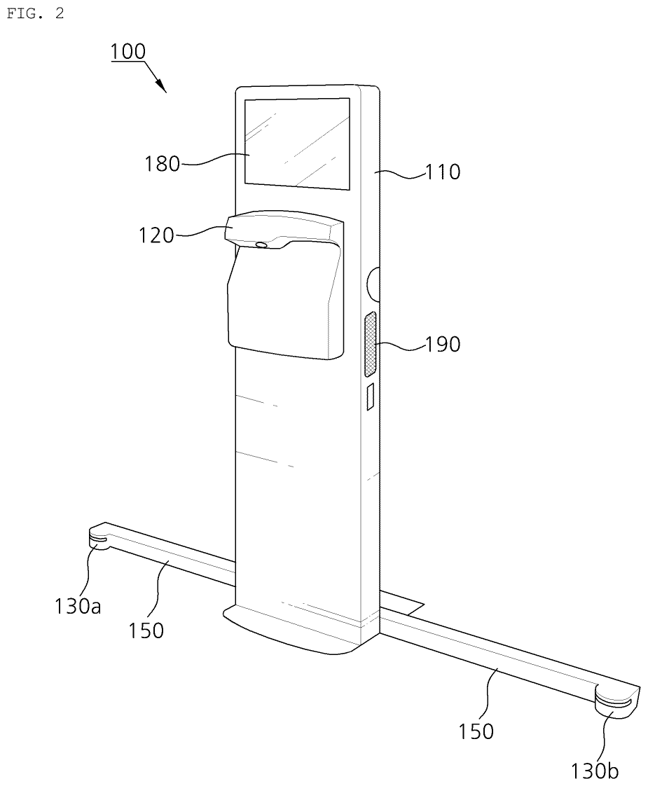

FIG. 2 is a view illustrating an appearance of the augmented reality interactive sports device 100 used in the physical fitness measurement method using the augmented reality interactive sports according to the exemplary embodiment of the present device 100 disclosure. As shown in FIG. 2, the augmented reality interactive sports device 100 may be configured to further include: a main body 110; a projector unit 120 provided on a front surface of the main body 110 and configured to project an image for sports content on a floor surface; LiDAR sensor units 130a and 130b installed toward a projection direction of the projector unit 120; a motion tracking unit (not shown) configured to recognize a user's position according to the sports content by using the LiDAR sensor units 130a and 130b and track the user's motion; a sensor connection unit 150 provided with first ends thereof respectively connected to side surfaces of a lower end of the main body 110 and second ends thereof respectively connected to the LiDAR sensor units 130a and 130b, so as to connect the LiDAR sensor units 130a and 130b to be spaced apart from the main body 110 by a predetermined distance; and a control unit (not shown) configured to provide the sports content to the user on the basis of augmented reality by outputting the sports content through the projector unit 120 and control interaction between the sports content and the user by using the user's motion tracked by the motion tracking unit (not shown). The augmented reality interactive sports device 100 may be configured to further include: a display unit 180 provided on the front surface of the main body 110 and configured to receive a selection signal for the sports content and output play information related to the sports content; and a speaker unit 190 configured to output sounds related to the sports content.

Here, the LiDAR sensor units 130a and 130b include two or more LiDAR sensors respectively connected to both sides of the lower end of the main body 110 through the sensor connection unit 150, so that a position of each user may be determined by avoiding tracking failure in a shadow area of laser-scanning, caused by relative positions of a plurality of users. In addition, the motion tracking unit 140 may recognize the user's foot positions by using the LiDAR sensor units 130a and 130b to perform laser scanning of a plane at a predetermined height from the ground, and track the user's motion by tracking changes in the user's foot positions.

The details of the augmented reality interactive sports device 100 may be configured by referencing Korean Patent No. 10-2233395 (invention title: AUGMENTED REALITY INTERACTIVE SPORTS APPARATUS USING LIDAR SENSORS, registration date: Mar. 23, 2021), Korean Patent No. 10-2430084 (invention title: AUGMENTED REALITY INTERACTIVE SPORTS SYSTEM USING LIDAR SENSORS, registration date: Aug. 2, 2022), etc.

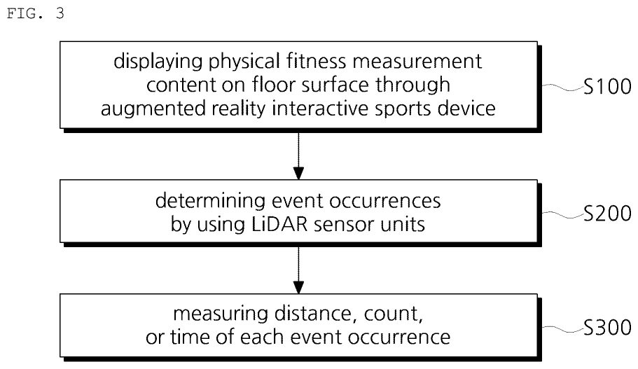

FIG. 3 is a view illustrating a flow of the physical fitness measurement method using the augmented reality interactive sports device 100 according to the exemplary embodiment of the present disclosure. As shown in FIG. 3, according to the exemplary embodiment of the present disclosure, the physical fitness measurement method using the augmented reality interactive sports device 100 may be realized by the augmented reality interactive sports device 100 that projects an image on a floor surface, tracks a user's motion by using LiDAR sensor units 130a and 130b, and controls interaction between content and the user, and may be realized to include: step S100 of displaying physical fitness measurement content on the floor surface through the augmented reality interactive sports device 100; step S200 of determining event occurrences by using the LiDAR sensor units 130a and 130b; and step S300 of measuring a distance, count, or time of each event occurrence.

In step S100, distance measurement-type, count measurement-type, or time measurement-type physical fitness measurement content may be displayed on a surface floor through the augmented reality interactive sports device 100. That is, as shown in FIG. 2, on the front surface thereof, the augmented reality interactive sports device 100 may be provided with a projector unit 120 for projecting a content image on the floor surface, and in step S100, the physical fitness measurement content may be projected and displayed onto the floor surface through the projector unit 120. In this case, the physical fitness measurement content may be of a distance measurement type, a count measurement type, or a time measurement type, and the content selected by a user may be displayed.

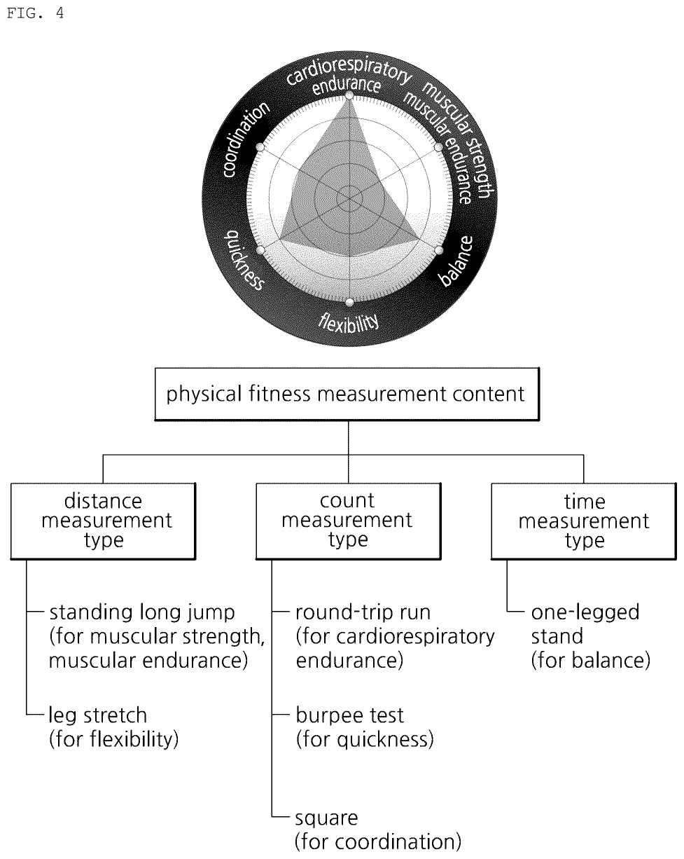

FIG. 4 is a view illustrating types of physical fitness measurement content in the physical fitness measurement method using the augmented reality interactive sports device 100 according to the exemplary embodiment of the present disclosure. As shown in FIG. 4, in the physical fitness measurement method using the augmented reality interactive sports device 100 according to the exemplary embodiment of the present disclosure, the physical fitness measurement content may be of a distance measurement type, a count measurement type, and a time measurement type. Physical fitness assessment standards consist of six categories: cardiorespiratory endurance, muscular strength/muscular endurance, balance, flexibility, quickness, and coordination. The physical fitness measurement content may be composed to include six types of sport events for assessing respective standards. As shown in FIG. 4, the six types of sport events may consist of a standing long jump (for muscular strength/muscular endurance), a leg stretch (for flexibility), a round-trip run (for cardiorespiratory endurance), a burpee test (for quickness), a square (for coordination), and a one-legged stand (for balance). Depending on data being measured, each sport event may be classified into a distance measurement type, a count measurement type, and a time measurement type. More specifically, the standing long jump and leg stretch correspond to the distance measurement type because they measure a distance. The round-trip run, burpee test, and square correspond to the count measurement type because they measure a user's exercise count. The one-legged stand corresponds to the time measurement type because this measures a time spent standing on one foot. In addition, the physical fitness measurement content may further include various sport events for physical fitness assessment.

In step S200, the user's foot or hand positions may be detected by using the LiDAR sensor units 130a and 130b performing laser scanning of the plane at the predetermined height from the floor surface, and event occurrences may be determined according to the foot or hand positions in accordance with the physical fitness measurement content. More specifically, the LiDAR sensor units 130a and 130b of the augmented reality interactive sports device 100 may recognize the user's foot positions at a predetermined point from the ground, and the motion tracking unit (not shown) may track the user's motion by tracking changes in the user's foot positions. In this case, the LiDAR sensor units 130a and 130b are configured to scan a foot height, but depending on the details of the physical fitness measurement content, a hand position may also be detected when a hand is positioned at the foot height. That is, the LiDAR sensor units 130a and 130b may detect the positions of the user's body by performing the laser scanning of a plane at a height of about 5 cm from the ground, and may transmit position information such as the detected feet to the physical fitness measurement device 200. In particular, as shown in FIG. 2, since the LiDAR sensor units 130a and 130b are configured with two or more LiDAR sensors, two or more laser scanning areas may overlap within a sensing area, thereby detecting a position of each of the left and right feet of the user accurately.

In addition, in step S200, event occurrences may be determined according to the positions of the detected feet or hands and the physical fitness measurement content. In the physical fitness measurement content, a case where positions or areas of sprites projected on the floor surface correspond to positions of the detected feet or hands may be determined as the event occurrences. More specifically, a case where at least a part of areas of the sprites projected on the floor surface overlaps with areas of the detected feet or hands, or specific positions of the sprites correspond to the areas of the feet or hands, or a predetermined ratio or more of the areas of the feet or hands overlap with the areas of the sprites may be determined as the event occurrences.

In step S300, a distance, count, or time of each event occurrence determined according to the physical fitness measurement content may be measured. As described in FIG. 4, depending on whether the physical fitness measurement content is the distance measurement type, the count measurement type, or the time measurement type, a physical fitness measurement result may be obtained by measuring the distance, count, and time of each event occurrence.

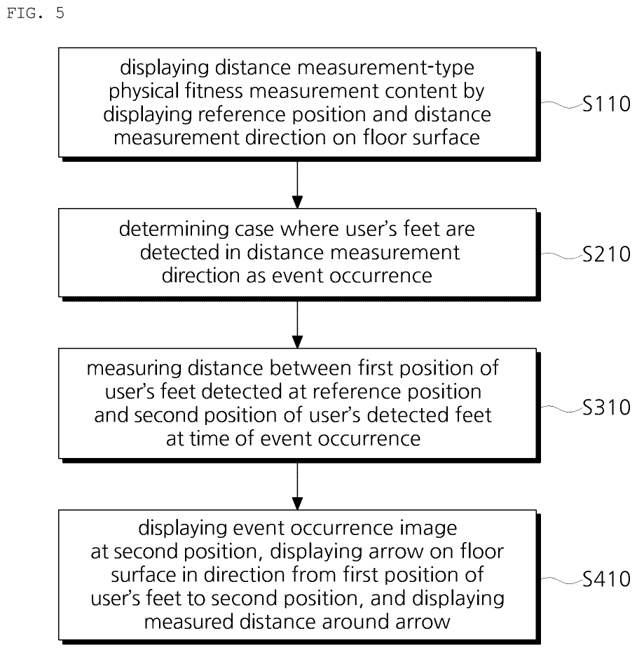

Hereinafter, with reference to FIGS. 5 to 13, physical fitness measurement content each having distance measurement type, count measurement type, and time measurement type will be described.

FIG. 5 is a view illustrating a flow of a physical fitness measurement method according to distance measurement-type physical fitness measurement content in the physical fitness measurement method using the augmented reality interactive sports device 100 according to the exemplary embodiment of the present disclosure. As shown in FIG. 5, in the physical fitness measurement method using the augmented reality interactive sports device 100 according to the exemplary embodiment of the present disclosure, when the distance measurement-type physical fitness measurement content is used, the physical fitness measurement method may be realized to include: step S110 of displaying distance measurement-type physical fitness measurement content by displaying a reference position and a distance measurement direction on a floor surface; step S210 of determining a case where a user's feet are detected in the distance measurement direction as an event occurrence; and step S310 of measuring a distance between a first position of the user's feet detected at the reference position and a second position of the user's detected feet at a time of the event occurrence. After step S310, the physical fitness measurement method may be realized to further include step S410 of displaying an event occurrence image at the second position, displaying an arrow on the floor surface in a direction from the first position of the user's feet to the second position, and displaying the measured distance around the arrow.

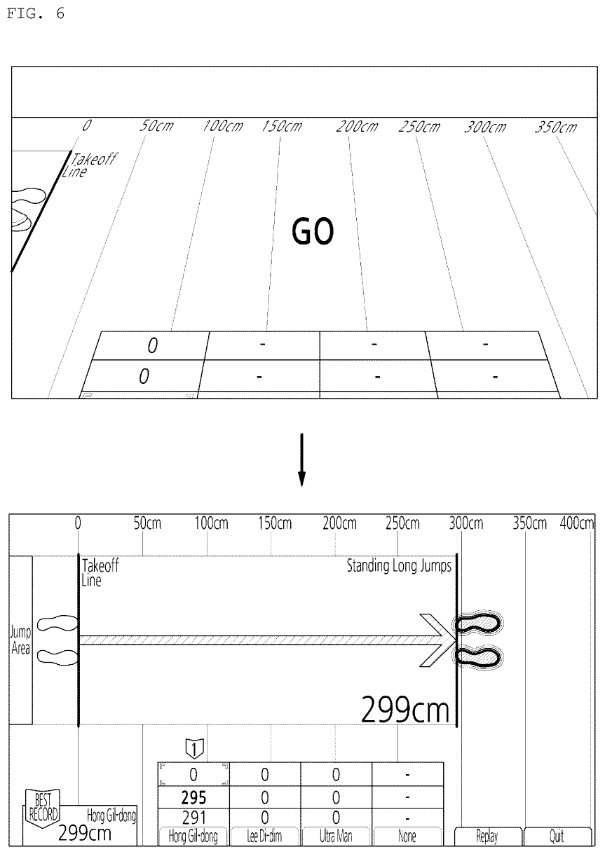

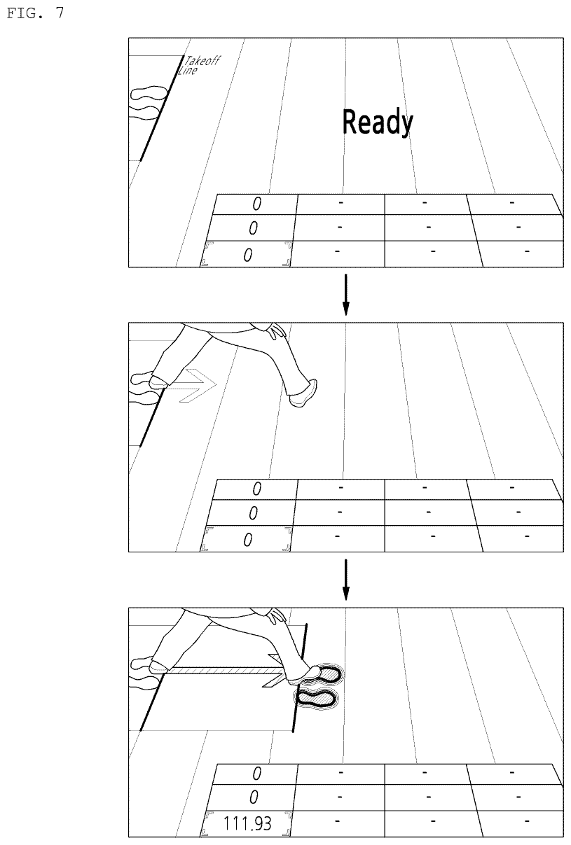

FIG. 6 is a view illustrating an example of an appearance of standing long jump content displayed on a floor surface in the physical fitness measurement method using the augmented reality interactive sports device 100 according to the exemplary embodiment of the present disclosure. FIG. 7 is a view illustrating an example of an appearance of leg stretch content displayed on a floor surface in the physical fitness measurement method using the augmented reality interactive sports device 100 according to the exemplary embodiment of the present disclosure. Hereinafter, specific steps for measuring a user's physical fitness through distance measurement-type physical fitness measurement content will be described in detail with reference to FIGS. 5 to 7.

In step S110, distance measurement-type physical fitness measurement content may be displayed by displaying a reference position and a distance measurement direction on a floor surface. Here, the reference position may be composed of a line, an image having a shape of two feet, or the like and the distance measurement direction may be indicated by an arrow or the like. However, when a user is enabled to intuitively identify the reference position and distance measurement direction at a time of performing distance measurement, the reference position and distance measurement direction may be expressed in various ways.

In step S210, a case where the user's feet are detected in the distance measurement direction may be determined as an event occurrence. In some cases, not only the user's feet but also hands, buttocks, etc. may be detected.

In step S310, a distance between a first position of the user's feet detected at the reference position and a second position of the user's feet detected at the time of the event occurrence is measured, so that any one selected from a group including a standing long jump distance and a leg stretch distance may be measured. That is, physical fitness may be measured by measuring the distance between the reference position presented in step S110 and the position where the event occurred.

In step S410, an event occurrence image may be displayed at the second position, an arrow may be displayed on the floor surface in a direction from the first position of the user's feet to the second position, and the distance measured in step S310 may be displayed around the arrow. Here, the event occurrence image may be diverse, such as an image having a shape of two feet, through which the user may visually estimate the measured distance. In addition, the arrow pointing from the reference position to the second position may be displayed as animation between the reference position and the second position, and the measured distance may be displayed on the floor surface together. As described above, by displaying the event occurrence image, arrow, measurement value, etc., a sense of trust may be given to the user and motivation for autonomous and repetitive measurements may be provided.

As shown in FIG. 6, in the standing long jump content, a white line on the left represents a reference line, and an image having a shape of two feet in red is displayed adjacent to the reference line, thereby enabling the user to intuitively recognize the reference position. In a case where the user stands at the reference position and performs a standing long jump toward the distance measurement direction, the user's body is detected in the distance measurement direction from the reference position, and this case may be determined as an event occurrence. The standing long jump involves jumping with two feet, but since the user may fall, various body parts such as the user's feet, buttocks, and hands may be detected in some cases. In this case, among objects detected in the distance measurement direction from the reference position, a position of an object closest to the reference position may be detected as an event occurrence position. The closest distance between the user's first position (i.e., the reference position) detected in a ready posture and the second position at the time of the event occurrence detected when the user performed the standing long jump and then landed may be measured. As shown in the second picture of FIG. 6, the second position detected at the time of the event occurrence may be visually displayed by displaying an image having a shape of two feet (i.e., an event occurrence image) at the second position. In this case, the arrow pointing from the reference position to the second position may be displayed as animation between the reference position and the second position.

As shown in FIG. 7, in the leg stretch content, a white line on the left represents a reference line, and a user may perform a leg stretch by placing one foot at a reference position and moving the other foot toward a distance measurement direction. In this case, the distance measurement direction may be indicated with an arrow, as shown in the middle picture of FIG. 7. Here, the arrow is composed of a moving arrow, so that the user is enabled to intuitively identify a long jump direction, a leg stretch direction, etc. In a case where the user completes the leg stretch and it is detected that the user's foot is stopped for a predetermined time or more in the distance measurement direction from the reference position, this case may be determined as an event occurrence. At this time, a case where both feet are respectively detected at the reference position and a position spaced apart from the reference position in the distance measurement direction may be determined as an event occurrence. A stretching distance is measured by measuring the reference position and a distance between the two feet that are detected at the time of the event occurrence, and an image having a shape of two feet (i.e., an event occurrence image) is displayed at the second position, and also an animation arrow, a measurement distance, etc. may be displayed together.

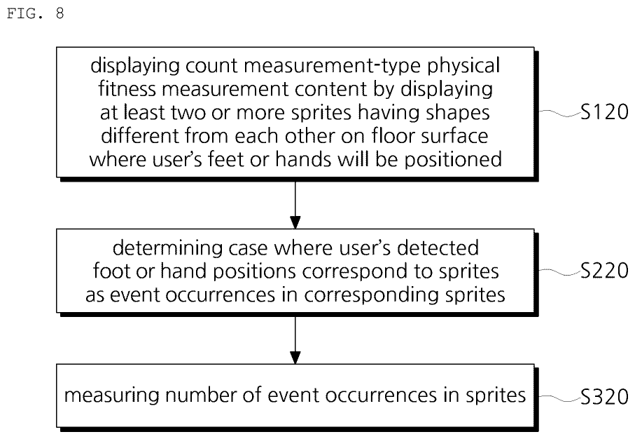

FIG. 8 is a view illustrating a flow of a physical fitness measurement method according to count measurement-type physical fitness measurement content in the physical fitness measurement method using the augmented reality interactive sports device 100 according to the exemplary embodiment of the present disclosure. As shown in FIG. 8, in the physical fitness measurement method using the augmented reality interactive sports device 100 according to the exemplary embodiment of the present disclosure, when the count measurement-type physical fitness measurement content is used, the physical fitness measurement method may be realized to include: step S120 of displaying count measurement-type physical fitness measurement content by displaying a first sprite and a second sprite, which have shapes different from each other, at first and second positions on a floor surface where a user's feet or hands will be positioned; step S220 of determining a case where the user's detected foot or hand positions correspond to the sprites as event occurrences in the corresponding sprites; and step S320 of measuring the number of event occurrences in the first sprite and the second sprite.

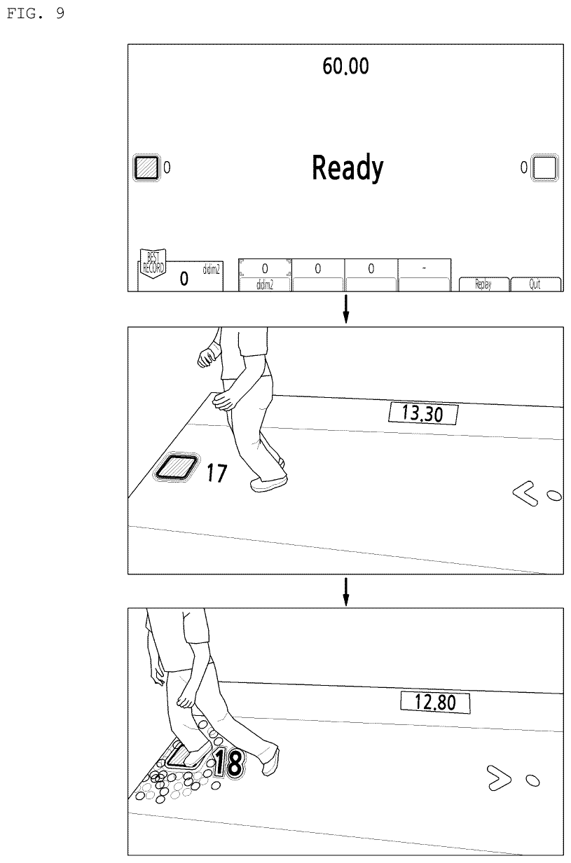

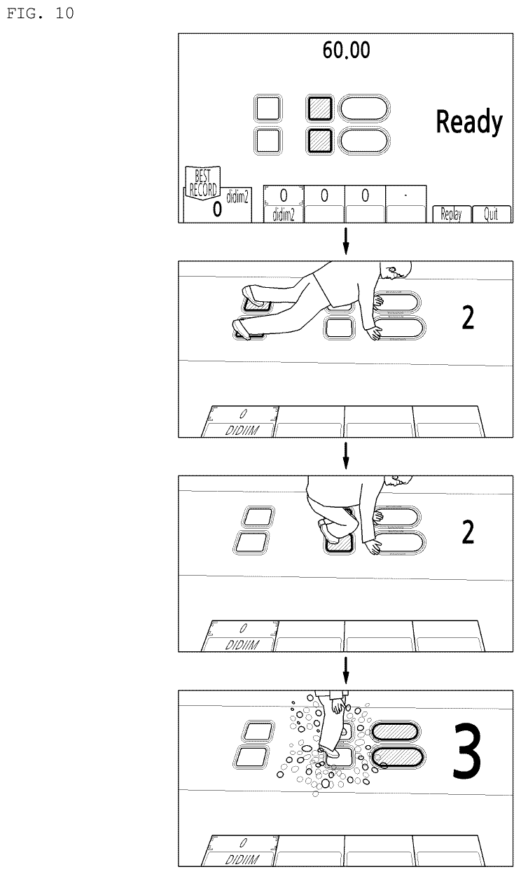

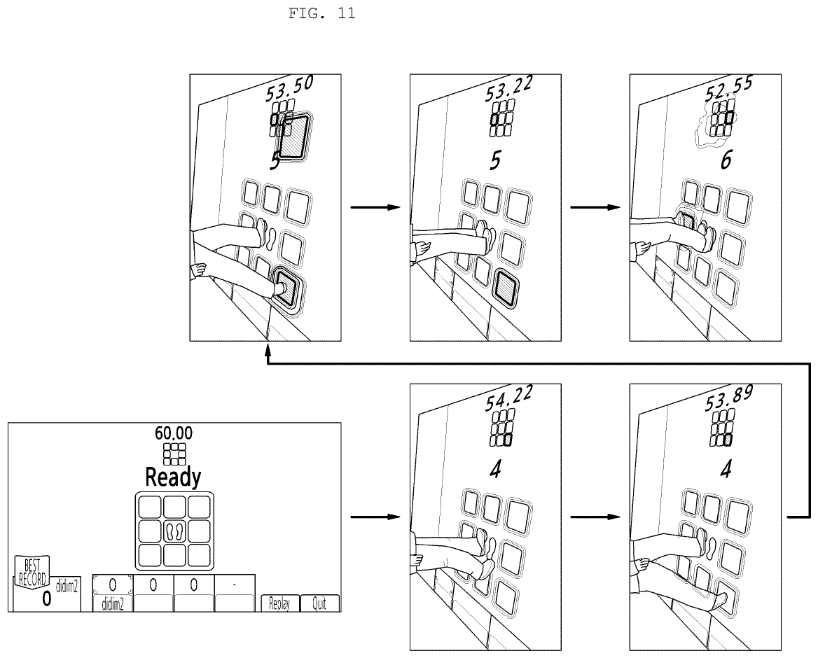

FIG. 9 is a view illustrating an example of an appearance of round-trip run content displayed on a floor surface in the physical fitness measurement method using the augmented reality interactive sports device 100 according to the exemplary embodiment of the present disclosure. FIG. 10 is a view illustrating an example of an appearance of burpee test content displayed on the floor surface in the physical fitness measurement method using the augmented reality interactive sports device 100 according to the exemplary embodiment of the present disclosure. FIG. 11 is a view illustrating an example of an appearance of square content displayed on the floor surface in the physical fitness measurement method using the augmented reality interactive sports device 100 according to the exemplary embodiment of the present disclosure. Hereinafter, specific steps for measuring a user's physical fitness through count measurement-type physical fitness measurement content will be described in detail with reference to FIGS. 8 to 11.

In step S210, at least two or more sprites with shapes different from each other are displayed on a floor surface where a user's feet or hands will be positioned, so that the count measurement-type physical fitness measurement content may be displayed. Here, each sprite is a two-dimensional graphic or object, which is synthesized into an image in computer graphics and is an object capable of moving independently of a background thereof. In step S210, at least two or more sprites different in at least one of forms, shapes, and colors thereof are displayed, so that the user is enabled to intuitively distinguish each sprite and understand the method of performing the physical fitness measurement content.

In step S220, a case where the user's detected foot or hand positions correspond to the sprites may be determined as event occurrences in the corresponding sprites. More specifically, in step S220, at least one of the forms, shapes, and colors of the sprites determined as to have the event occurrences may be displayed to change, so as to indicate that the user's feet or hands correspond to the corresponding sprites.

In step S230, the number of event occurrences in the sprites may be measured. More specifically, in step S230, the number of event occurrences during a predetermined time period may be measured, and any one selected from a group including the number of round-trip runs and the number of burpee tests may be measured.

As shown in FIG. 9, in the round-trip run content, sprites having colors different from each other may be arranged at both ends of a floor surface on which the content is projected. To help users play, a direction for running may be indicated by displaying a moving arrow and the like between the two sprites. The user may run back and forth between the two sprites and step on each sprite, thereby causing events to occur when the sprites and positions of the user's feet correspond to each other. In this case, as shown in the last picture of FIG. 9, when an event occurs, a form, shape, color, etc.

of each sprite may be changed. In addition, the number of event occurrences occurring in the respective sprites may be displayed as a number around the corresponding sprites. When the user steps on a blue sprite in the second picture of FIG. 9, the user may confirm that a size, shape, and color of the corresponding sprite temporarily change, and the number of event occurrences increases from 17 to 18, as shown in the third picture of FIG. 9.

As shown in FIG. 10, in the burpee test content, sprites for hands and feet may be composed in shapes different from each other, and sprites for first and second positions of the feet may be composed in colors different from each other. A user may perform one burpee test by placing his or her hands on respective oval sprites, moving in sequence his or her feet to blue square sprites, white square sprites, and the blue square sprites again, then removing the hands from a floor surface and standing up. In this case, event occurrences (i.e., performing a burpee test) may be determined by combining the sequence of feet or hands detection in three types of the sprites, and thus the number of event occurrences may be measured. As shown in the last picture of FIG. 10, it may be seen that when the user performs the burpee test once, the number of event occurrences, which is two, displayed around the sprites increases to three.

As shown in FIG. 11, in the square content, in a condition where a square is divided into 3*3 spaces, a central reference position and the remaining eight spaces are composed of sprites of shapes different from each other, so that a position of a sprite that has to be stepped on at a reference position (in a shape of two feet) may be presented as an image. A user may perform physical fitness measurement by looking at the presented image and stepping on the corresponding sprite.

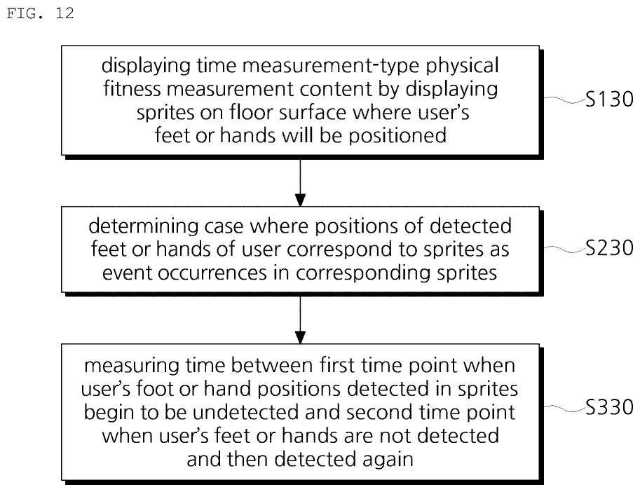

FIG. 12 is a view illustrating a flow of a physical fitness measurement method according to time measurement-type physical fitness measurement content in the physical fitness measurement method using the augmented reality interactive sports device 100 according to the exemplary embodiment of the present disclosure. As shown in FIG. 12, in the physical fitness measurement method using the augmented reality interactive sports device 100 according to the exemplary embodiment of the present disclosure, when the time measurement-type physical fitness measurement content is used, the physical fitness measurement method may be realized to include: step S130 of displaying time measurement-type physical fitness measurement content by displaying sprites on a floor surface where a user's feet or hands will be positioned; step S230 of determining a case where positions of the detected feet or hands of the user correspond to the sprites as event occurrences in the corresponding sprites; and step S330 of measuring a time between a first time point when the user's foot or hand positions detected in the sprites correspond to the sprites and a second time point when the user's feet or hands corresponding to the sprites are not detected and then detected again.

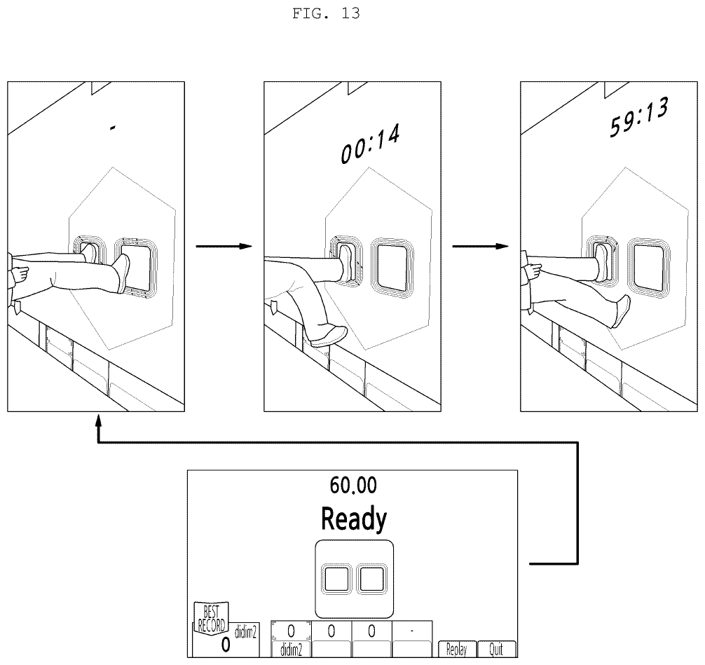

FIG. 13 is a view illustrating an example of an appearance of one-legged stand content displayed on a floor surface in the physical fitness measurement method using the augmented reality interactive sports device 100 according to the exemplary embodiment of the present disclosure. Hereinafter, specific steps for measuring a user's physical fitness through time measurement-type physical fitness measurement content will be described in detail with reference to FIGS. 12 and 13.

In step S310, time measurement-type physical fitness measurement content may be displayed by displaying sprites on a floor surface where a user's feet or hands will be positioned. That is, in one-leg stand content, positions for both feet may be displayed as square-shaped sprites, as shown in the first picture of FIG. 13.

In step S320, a case where the user's detected foot or hand positions correspond to the sprites may be determined as event occurrences in the corresponding sprites. More specifically, in step S320, at least one of sizes, shapes, and colors of the sprites determined as to have the event occurrences may be displayed to change, so as to indicate that the user's feet or hands correspond to the sprites. That is, as shown in the second and third pictures of FIG. 13, a sprite where an event occurs due to stepping on the sprite may be composed, so as to allow two blue bands to rotate along the edge of the sprite.

In step S330, a time between a first time point when the user's foot or hand positions detected in the sprites begin to be undetected and a second time point when the user's feet or hands are not detected and then detected again may be measured, so that any one selected from a group including one-legged stand time may be measured. In this case, in step S330, the user's feet or hands detected at the second time point do not necessarily correspond to the sprites, and within a preset range in the corresponding sprites, a time point at which the user's hands or feet are detected may become the second time point. That is, in the second picture of FIG. 13, the right foot is detected at a position corresponding to a right sprite. A time point at which only one of the two feet is detected due to the lifted right foot may become the first time point. In the third picture of FIG. 13, the right foot is not detected, but in the fourth picture, the right foot is detected again in a place other than the right sprite. In this case, in the one-legged stand content, even when the right foot is not on the right sprite, a time point 59:13, which is a time point when the right foot is detected again (in the fourth picture), may be detected as the second time point.

As described above, according to the physical fitness measurement method using the augmented reality interactive sports device 100 proposed in the present disclosure, there is an effect of displaying distance measurement-type, count measurement-type, or time measurement-type physical fitness measurement content on a floor surface through the augmented reality interactive sports device and determining event occurrences according to foot or hand positions by using LiDAR sensors, so as to measure a distance, count, or time of each event occurrence, whereby a user's physical fitness may be conveniently measured by using digital technology, physical fitness may be repeatedly and autonomously measured, and changes in the physical fitness may be monitored. In addition, According to the present disclosure, there is another effect of performing laser scanning of heights of foot positions at a certain height on a floor surface, so as to determine event occurrences, thereby measuring lower body abilities such as a long jump, a round-trip run, etc. in an easy and fun way.

Meanwhile, the embodiment of the present disclosure may include a computer-readable medium including program instructions for performing operations implemented in various communication terminals. For example, the computer-readable media may include hardware devices, which are specially configured to store and execute program instructions, that include: magnetic media such as hard disks, floppy disks, and magnetic tapes; optical media such as CD ROMs and DVDs; magneto-optical media such as floptical disks; and memories such as ROMS, RAMs, and flash memories.

As such, the computer-readable media may include program instructions, data files, data structures, and the like individually or in combination. In this case, the program instructions recorded on the computer-readable media may be specially designed and configured to implement the embodiment of the present disclosure, or may be known and available to those skilled in the art of computer software. For example, the computer instructions may include not only machine language code such as one generated by a compiler, but also high-level language code executable by a computer using an interpreter or the like.

The above description of the present disclosure is for illustration, and it will be understood that those skilled in the art to which the present disclosure pertains may easily transform the present disclosure in other specific forms without departing from the technical spirit or essential features thereof. Therefore, it should be understood that the above-described exemplary embodiment is illustrative in all respects and not restrictive. For example, each component described as a single type may be implemented in a distributed manner, and similarly, components described as distributed may be implemented in a combined form.

The scope of the present disclosure is indicated by the following claims rather than the above detailed description, and all changes or modifications derived from the meaning and scope of the claims and equivalent concepts should be interpreted as being included in the claims of the present disclosure.

What is claimed is:

1. A physical fitness measurement method using an augmented reality interactive sports device (100), the physical fitness measurement method being realized by the augmented reality interactive sports device (100) that projects an image on a floor surface, tracks a user's motion by using LiDAR sensor units (130a and 130b), and controls interaction between content and the user, and the physical fitness measurement method comprising:
step (1) of displaying distance measurement-type, count measurement-type, or time measurement-type physical fitness measurement content on the floor surface through the augmented reality interactive sports device (100);
step (2) of using the LiDAR sensor units (130a and 130b) that perform laser scanning of a plane at a predetermined height from the floor surface, so as to detect foot or hand positions of the user and determining event occurrences according to the physical fitness measurement content and the foot or hand positions; and
step (3) of measuring a distance, count, or time of each event occurrence determined according to the physical fitness measurement content.

2. The physical fitness measurement method of claim 1, wherein a reference position and a distance measurement direction are displayed on the floor surface, so as to display the distance measurement-type physical fitness measurement content in step (1),
each event occurrence is determined when the user's feet are detected in the distance measurement direction in step (2), and
a distance between a first position of the user's feet detected at the reference position and a second position of the user's feet detected at a time of each event occurrence is measured, so that any one selected from a group comprising a standing long jump distance and a leg stretch distance is measured in step (3).

3. The physical fitness measurement method of claim 2, further comprising:
after step (3), step (4) of displaying an event occurrence image at the second position, displaying an arrow on the floor surface in a direction from the first position of the user's feet to the second position, and displaying the distance measured in step (3) around the arrow.

4. The physical fitness measurement method of claim 1, wherein at least two or more sprites having shapes different from each other are displayed on the floor surface where the user's feet or hands will be positioned, so as to display the count measurement-type physical fitness measurement content in step (1),
the event occurrences are determined in the corresponding sprites when the user's detected foot or hand positions correspond to the sprites in step (2), and
the number of event occurrences in the sprites is measured in step (3).

5. The physical fitness measurement method of claim 4, wherein, in step (2), at least one of forms, shapes, and colors of the sprites determined as to have the event occurrences is displayed to change, so as to indicate that the user's feet or hands correspond to the corresponding sprites.

6. The physical fitness measurement method of claim 4, wherein, in step (3), the number of event occurrences during a predetermined time period is measured, and
any one selected from a group comprising the number of round-trip runs and the number of burpee tests is measured.

7. The physical fitness measurement method of claim 1, wherein sprites are displayed on the floor surface where the user's feet or hands will be positioned, so as to display the time measurement-type physical fitness measurement content in step (1),
the event occurrences s are determined in the corresponding sprites when the user's detected foot or hand positions correspond to the sprites in step (2), and
a time between a first time point when the user's detected foot or hand positions in the sprites begin to be undetected and a second time point when the user's feet or hands are not detected and then detected again is measured, so as to measure any one selected from a group comprising a one-legged stand time in step (3).

8. The physical fitness measurement method of claim 7, wherein, in step (2), at least one of sizes, shapes, and colors of the sprites determined as to have the event occurrences is displayed to change, so as to indicate that the user's feet or hands correspond to the corresponding sprites.
